(19) United States
(12) Patent Application Publication
(10) Pub. No.: US 2014/0102449 A1
(43) Pub. Date: Apr. 17, 2014

(54) MOUNTING UNIT FOR A BLOWER DEVICE AND SYSTEM FOR INTERCHANGING A BLOWER DEVICE BETWEEN VARIOUS MOUNTING UNITS

(71) Applicant: Michael G. Lalonde, Alpharetta, GA (US)

(72) Inventor: Michael G. Lalonde, Alpharetta, GA (US)

(73) Assignee: Deshum Medical, LLC, Cambridge, MA (US)

(21) Appl. No.: 13/873,152

(22) Filed: Apr. 29, 2013

Related U.S. Application Data
(63) Continuation-in-part of application No. 13/450,614, filed on Apr. 19, 2012.
(60) Provisional application No. 61/639,914, filed on Apr. 28, 2012.

(30) Foreign Application Priority Data
Oct. 20, 2010 (US) PCT/US2010/005337

(51) Int. Cl. A61M 16/00; A61M 16/06; A61M 16/08

(52) U.S. Cl. CPC A61M 16/0066; A61M 16/0816; A61M 16/0051; A61M 16/0683; A61M 16/0875
USPC 128/202.27; 128/204.18; 128/204.21; 128/205.25

(57) ABSTRACT

A mounting unit for use with a portable blower device and a system of a simple portable blower device that is interchangeable with a plurality of mounting devices. The portable blower device has a gas flow generator for providing a flow of gas to a mask for delivery of gas flow to an airway of a patient.

### CROSS-REFERENCE TO RELATED APPLICATIONS

This application is a Continuation-in-Part of U.S. Ser. No. 13/450,614 filed Apr. 19, 2012, which claims the benefit of PCT Application PCT/US2010/053370 filed on Oct. 20, 2010 which claims the benefit of U.S. Patent Application 61/253,500 filed on Oct. 20, 2009, U.S. Patent Application 61/288,290 filed on Dec. 19, 2009, and U.S. Patent Application 61/301,151 filed on Feb. 3, 2010, and this application claims the benefit of U.S. Patent Application 61/639,914 filed Apr. 28, 2012, which are incorporated herein by reference.

### COPYRIGHT INFORMATION

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates to a continuous positive airway pressure (CPAP) machine and more particularly to a CPAP machine that has a first unit that can be placed in a second unit.

### BACKGROUND OF THE INVENTION

Sleep apnea syndrome afflicts an estimated 1% to 5% of the general population and is due to episodic upper airway obstruction during sleep. Those afflicted with sleep apnea experience sleep fragmentation and intermittent, nearly complete, or complete, cessation of ventilation during sleep with potentially severe degrees of oxyhemoglobin de-saturation.

Although details of the pathogenesis of upper airway obstruction in sleep apnea patients have not been fully defined, it is generally accepted that the mechanism includes either anatomic or functional abnormalities of the upper airway which result in increased air flow resistance. Such abnormalities may include narrowing of the upper airway due to suction forces evolved during inspiration, the effect of gravity pulling the tongue back to oppose the pharyngeal wall, and/or insufficient muscle tone in the upper airway dilator muscles. It has also been hypothesized that a mechanism responsible for the known association between obesity and sleep apnea is excessive soft tissue in the anterior and lateral neck which applies sufficient pressure on internal structures to narrow the airway.

Recent work in the treatment of sleep apnea has included the use of continuous positive airway pressure (CPAP) to maintain the airway of the patient in a continuously open state during sleep. Unfortunately, the statistics on CPAP non-compliance are startling. There are numerous reasons for non-compliance including the lack of portability and noise levels.

### SUMMARY OF THE INVENTION

It has been recognized that users of a continuous positive airway pressure (CPAP) apparatus desire the ability to use the apparatus in various locations including home and on the road including hotels and airplanes. In addition, the user desires several features including a quiet system and a small portable system.

A continuous positive airway pressure (CPAP) system provides positive airway pressure therapy having a first unit which includes a compressor and a second unit that receives the first unit and includes at least a portion of the air pathway upstream of the compressor.

In an embodiment a gas delivery system provides positive airway pressure therapy during a user's sleep period; the system has a first unit and a second unit. The first unit has a compressor that pressurizes the gas. The compressor includes an impeller and a motor. The first unit has an input to the impeller for receiving air and an outlet for expelling compressed air. The second unit defines a cavity to receive the first unit and defines an air pathway through which air passes prior to the compressor in the first unit.

In an embodiment, the second unit is a hard case having a base having the cavity to receive the first unit and a cover to overlie the first unit.

In an embodiment, the second unit is a pouch having the cavity to receive the first unit. The pouch has a rigid portion defining the air pathway through which air passes prior to the compressor in the first unit.

In an embodiment, the second unit is incorporated into a mask.

In an embodiment, the second unit has a connection for engaging a connection on the first unit for transmitting electricity to the first unit; the second unit has a power source. In an embodiment, the power source is a battery.

In an embodiment of a gas delivery system that provides positive airway pressure therapy during a user's sleep period, the system has a first unit and a second unit. The first unit has a compressor that pressurizes the gas. The compressor includes an impeller and a motor. The first unit has an input to the impeller for receiving air and an outlet for expelling compressed air. The first unit has a controller for operating the compressor. The second unit defines a cavity to receive the first unit and defines an air pathway through which air passes prior to the compressor in the first unit.

In an embodiment, the second unit has a connection for engaging a connection on the first unit for transmitting electricity to the first unit. The second unit has a power source.

In an embodiment, the second unit has a port for allowing access to a receptacle carried on the first unit to receive electricity.

In an embodiment, the first unit has an input device for the operator to control the system.

In an embodiment, the second unit has an input device for the operator to control the system.

In an embodiment, the second unit has a plurality of input devices for the operator to control the system.

In an embodiment, the first unit further comprises at least one sensor for monitoring the system and providing input to the controller.

In an embodiment of a gas delivery system that provides positive airway pressure therapy during a user's sleep period, the system has a first unit and a second unit. The first unit has a compressor that pressurizes the gas, the compressor including an impeller and a motor. The first unit has an input to the impeller for receiving air and an outlet for expelling compressed air. The first unit has a controller for operating the compressor and at least one sensor for monitoring the system and providing input to the controller. The second unit defines a cavity to receive the first unit and defines an air pathway through which air passes prior to the compressor in the first unit. The second unit has a connection for engaging a connection on the first unit for transmitting electricity to the first unit.

In an embodiment, the first unit has a data storage device for recording data from the system.

In an embodiment, the second unit has a data storage device for recording data from the system.

In an embodiment, the system has a remote control port having an input device for the operator to control the system. In an embodiment, the remote control interfaces with the second unit via Bluetooth.

These aspects of the invention are not meant to be exclusive and other features, aspects, and advantages of the present invention will be readily apparent to those of ordinary skill in the art when read in conjunction with the following description, appended claims, and accompanying drawings.

### BRIEF DESCRIPTION OF THE DRAWINGS

The foregoing and other objects, features, and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic of a CPAP system with a sleep activity control system with a first unit including a compressor that is received by a second unit including an acoustic suppressor;

FIG. 2 is a top view of a blower unit, a first unit;

FIG. 3 is an isometric view of the blower unit with a portion removed;

FIG. 4 is a sectional view of the blower unit taken along line 4-4 of FIG. 2;

FIG. 5 is a perspective view of the docking station;

FIG. 6A is a top view of the docking station of FIG. 5;

FIG. 6B is a front view of the docking station of FIG. 5;

FIG. 6C is a side view of the docking station;

FIG. 7 is a perspective view of the docking station with the upper clam shell hinged upward;

FIG. 8A is a top view of an alternative docking station;

FIG. 8B is the side view of the alternative docking station of FIG. 8A;

FIG. 9 is a perspective view of a pouch, a second unit, adapted for receiving the blower unit, a first unit;

FIG. 10 is a top view of a pouch of FIG. 9;

FIG. 11 is a side view of the system on a user;

FIG. 12 illustrates a perspective view of a portable CPAP device

FIG. 13 illustrates a perspective view of a portable CPAP device mounted in a cradle having a power source.

FIGS. 14A-D illustrate various views of a supple pouch having various attachment means.

### DETAILED DESCRIPTION OF THE INVENTION

A system and method for delivering pressurized gas to the airway of a patient, the system having a gas flow generator for providing a flow of gas and a mask for the delivery of the gas flow to an airway of a patient. The system has a unit that contains a compressor including a motor and impeller that is received in a second unit that receives the first unit and includes at least a portion of the air pathway upstream of the compressor. The second unit can take various forms including a clam shell adapted to be received on a table, a pouch, or a mask.

The abbreviation CPAP stands for continuous positive air pressure which in generic terms is a method of noninvasive or invasive ventilation assisted by a flow of air delivered at a positive pressure throughout the respiratory cycle. It is performed for patients who can initiate their own respirations but who are not able to maintain adequate arterial oxygen levels without assistance. Sometimes the word “continuous” is replaced with the “constant.” For the purpose of this patent, constant positive airway pressure is referred to as mono-level CPAP. CPAP can be in various modes including mono-level CPAP, Bi-level CPAP, Auto-PAP, Servo-ventilation, and ramping. The pressure can also be varied in each pressure mode mentioned to range from 0 pressures to 50 cm H₂O pressure.

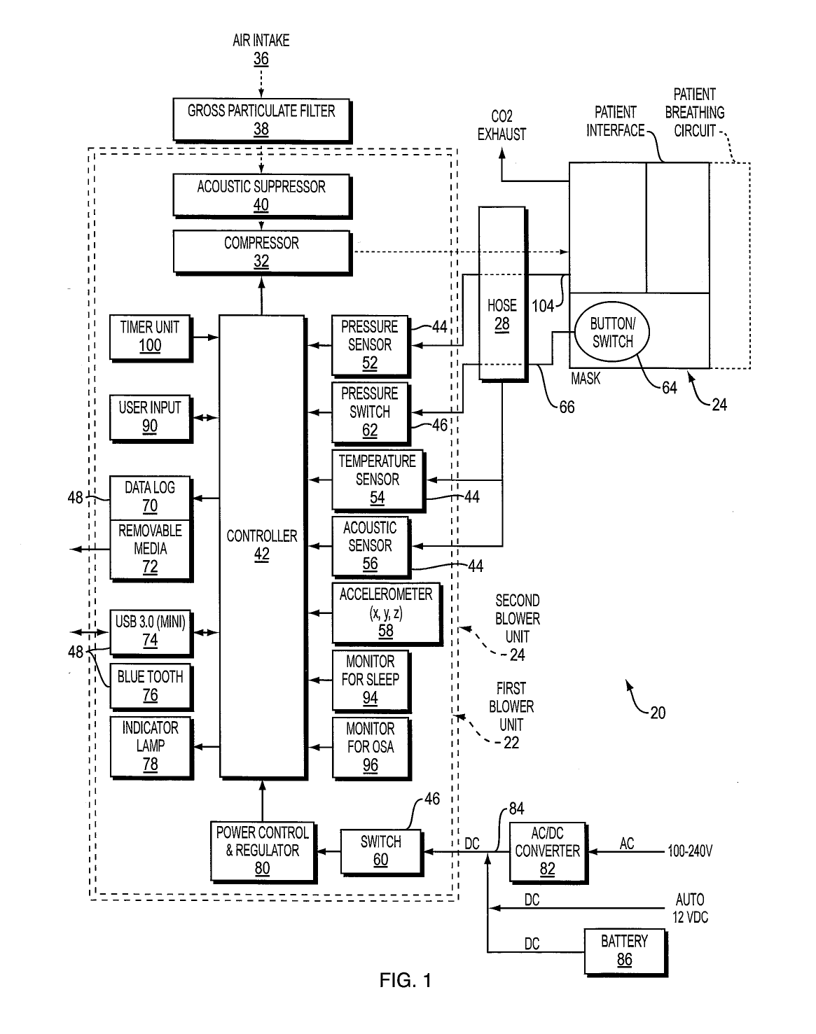

Referring to FIG. 1, a schematic of a CPAP system 20 with a first unit 22 that is received is a second unit 24 is shown. The first unit 22 is a blower unit 22, also referred to as a gas flow generator, for providing a source of pressurized breathable air, a patient interface 26, such as a mask, that is removably worn by the patient, and an interconnector 28, such as a hose. The first unit, the blower unit 22, is enclosed in a second unit 24. The first unit 22 has a compressor 32 for taking ambient air and creating pressurized air flow. The desired pressure range can vary, but generally falls in the range of between 4 and 20 centimeters of water. The average user/patient however requires between 6 and 14 centimeters of water. Air flow from the compressor can be adjusted for a rate of 20 to 60 liters of air per minute.

The air for the mask 26 is drawn in at an air intake 36 and passes through a filter 38 and an acoustic suppressor 40 both located in the second unit 24 prior to the blades of the impeller of the compressor 32. The compressor 32 compresses the air, thereby increasing the pressure; an expansion chamber of the compressor allows the compressed air to expand and increase the velocity of the air. The pressurized air passes through the interconnector 28 to the mask 26.

The blower unit 22, the first unit 22, in addition has a controller 42 and a plurality of sensors 44, switches 46, and interface devices 48 for controlling the compressor 32.

The sensors 44 can include a pressure sensor 52 that monitors the pressure of the air in the blower unit 22, the interconnector 28, and/or the mask 26. The sensors 44 can also include a temperature sensor 54, an acoustic sensor 56, and an accelerometer 58. The plurality of switches 46 includes a switch 60 for the system 20 located on the blower unit 22. In addition, the system 20 has a pressure switch 62 which connects to a switch 64 on the mask 26 with a conduit 66 carried by the interconnector 28.

The interface devices 48 include a data log 70 associated with removable media 72. The interface devices 48 can also include a USB port 74, blue tooth 76, and an indicator lamp 78.

Still referring to FIG. 1, the blower unit 22 has a power control and regulator 80 interposed between the switch 60 and the controller 42. The system 20 can be powered by various methods as represented by the AC/DC converter 82, a DC output 84 such as from an auto, and/or a battery 86. While 12 volts DC is shown, it recognized that the system may receive power inputs at different voltages such as 14-15 volts, 19 volts, or 24 volts.

The system 20 has a user input 90 that allows the user/clinician to select I modify the working of the system 20. For example, the clinician can adjust the pressures or mode of treatment. The mode could include mono-level CPAP, hi-level CPAP, and ramping. The user can select for example when the blower turns on as described in the paragraph below.

In addition, the blower (flow generator) unit 22 has a timer unit 100 that is capable of controlling when the compressor 32 is on and providing pressured air to the patient interface, mask 26 through the interconnector 28. In addition, the blower unit 22 in certain embodiments has an interface device 94 for detecting and monitoring sleep stages; as explained in more detail below, the interface device takes input from a sensor and determines if the user is asleep. In addition, in certain embodiments the blower unit 22 has a second or alternative interface device 96 for monitoring for detecting obstructed sleep apnea. The timer unit 100, the interface device 94 for detecting sleep stage, and the interface device 96 for detecting OSA are described in provisional application 61/559,912 filed on Nov. 15, 2011 which is incorporated herein by reference.

The mask 26 is most commonly a nasal mask or a full face mask as shown. It is recognized that the patient interface 26 can be other devices such as a nasal cannulae, an endotracheal tube, or any other interface, as explained below, based on other suitable appliances for interfacing between a source of breathing gas and a patient.

It is recognized that certain components that are shown in the first unit 22 can be moved to the second unit 24. In addition, the gross particulate filter 38 can be located in the first unit 22 in contrast to the second unit 24.

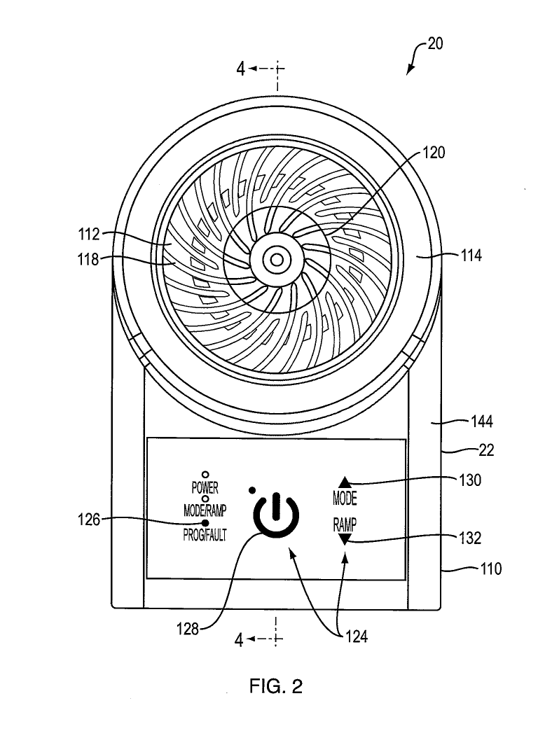

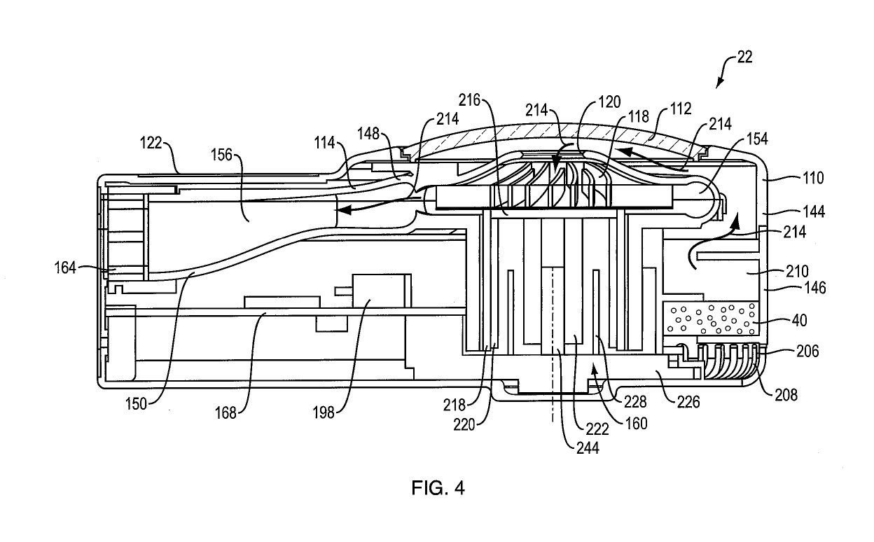

Referring to FIG. 2, a top view of the first unit 22, the blower unit I flow generator unit 22 is shown. The blower unit 22 as indicated above takes air and compresses the air to create a pressurized gas (air) that can be delivered to the patient interface 26, such as a mask at a pressure between 4 and 20 centimeters of water and at a flow rate of between 20 to 60 liters of air per minute in an embodiment. The blower unit 22 has a housing 110 with a translucent dome 112. In addition, the blower unit 22 has a casing 114, which in the embodiment shown is transparent, showing an impeller 118 of the compressor 32. The casing 114 has an opening 120 through which air flows as explained in greater detail with respect to FIG. 4. The blower unit 22 has a first input/output portion 122 which has a plurality of switches 124 and a plurality of indicators 126. In the embodiment shown, the first input/output portion 122 is a membrane switch 122 having three switches 128, 130, and 132 and four LED indicators 134, 136, 138, and 140.

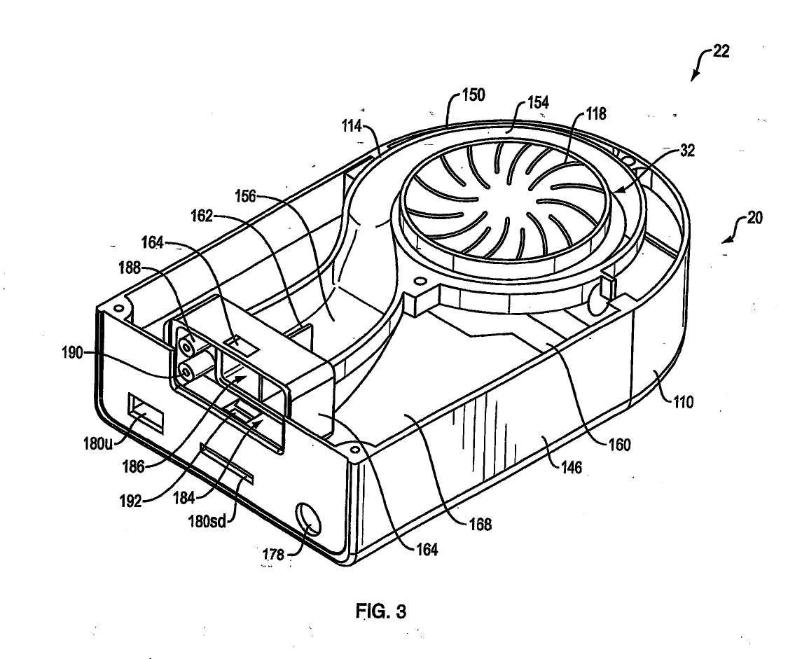

Referring to FIG. 3, an isometric view of the first unit, the blower unit 22 with a portion removed is shown. The housing 110 has an upper shell 144, seen in FIG. 2, which is removed in FIG. 3 and a lower shell 146. The casing 114 has an upper portion 148, which is removed in FIG. 3, and a lower portion 150. The casing 114 defines a collection chamber 154 of the compressor 32 which encircles the impeller 118. As the impeller 118 rotates counter-clockwise, the air is pushed into the collection chamber 154 and moves into an expansion chamber 156 as defined by the casing 114. Underlying the impeller 118 is a motor 160 of the compressor 32.

Still referring to FIG. 3, the casing 114 at the expansion chamber 156 end has a fin 162 that splits the flow of air into two portions. The housing 110 has a hose interface connector 164 that interfaces with the hose 28. The blower unit 22 has a printed circuit board (PCB) 168 that contains the circuitry to both monitor the inputs and control the motor 160. The PCB 168 has a variety of components including a motor control integrated circuit, an orientation sensor, a pressure switch, and a pressure sensor. In addition, mounted on the PCB 168 are a power connector 178 and a pair of data connectors 180 in the embodiment shown. The first data connector is a mini USB receptacle 180u and the second data connector is a micro sd card reader 180s.

The hose interface connector 164 of the housing 110 has a generally rectangular opening that receives the hose 28. The hose interface connector 164 has an opening 184 that opens up onto an air flow hole 186 that receives the end of the casing 114. In addition the connector 164 has a pair of projections 188 that are received by the hose 28. Each projection 188 has an opening 190 that is in communication with a sensor or switch. In addition, the hose interface connector 164 has a pair of detent openings 192 for securing the hose 28.

Referring to FIG. 4, a sectional view of the blower unit 22 taken along line 4-4 of FIG. 2 is shown. The housing 110 with the translucent dome 112 of the blower unit 22 encases the casing 114 that defines the collection chamber 154 and the expansion chamber 156. The end of the casing 114 is shown fitted into the hose interconnection connector 164. The PCB 168 has various components including a motor control integrated circuit, an orientation sensor 198, a pressure switch, and a pressure sensor.

The blower unit 22 has a series of slots 206 in the housing 110 defining an intake 208 through which it draws in ambient air. The air is drawn through a series of baffle chambers 160 defined by the shell 110 and used to suppress noise. A filter 38 is located in the baffle chamber 210 for blocking particulate that may be in the air. The air flows out of the baffle chamber 210 and between the casing 114 and the upper shell 144 including the translucent dome 112 and is drawn through the opening 120 in the casing 114. The impeller 118, which is enclosed in the casing 114, forces the air into the collection chamber 154 as it rotates. The collection chamber 154 increases in size as it encircles the impeller 118 in the counterclockwise direction. The pressurized air expands in the expansion chamber 156 as it moves to the hose interface connector 164. Arrows 214 show the flow of the air through the blower unit 22.

The motor 160 that drives the impeller 118 has an upper portion 216 with an outer sleeve 218 that encircles a magnet 220. The upper portion 216 is held in position by an air bearing sleeve 222 encircling a pin 224 projecting upward from a motor board 226. The motor board also has a careless waveform continuation coil 228 that receives current in a manner that creates a field to influence the magnet and rotates the upper portion 216 of the motor and the impeller 118.

In an embodiment, the blower unit 22 is approximately 4 inches by 2.5 inches by 1.5 inches in size. The weight of the blower unit 22 is less than 8 ounces.

When the user is ready to use the CPAP system 20, the user turns on the system 20 by turning on the switch as represented by block 60 in FIG. 1. This action places the unit into a stand-by mode. The system 20 can operate in several different modes. While some of the operations are described separately, it is recognized that one or more of the modes of operation can be used concurrently.

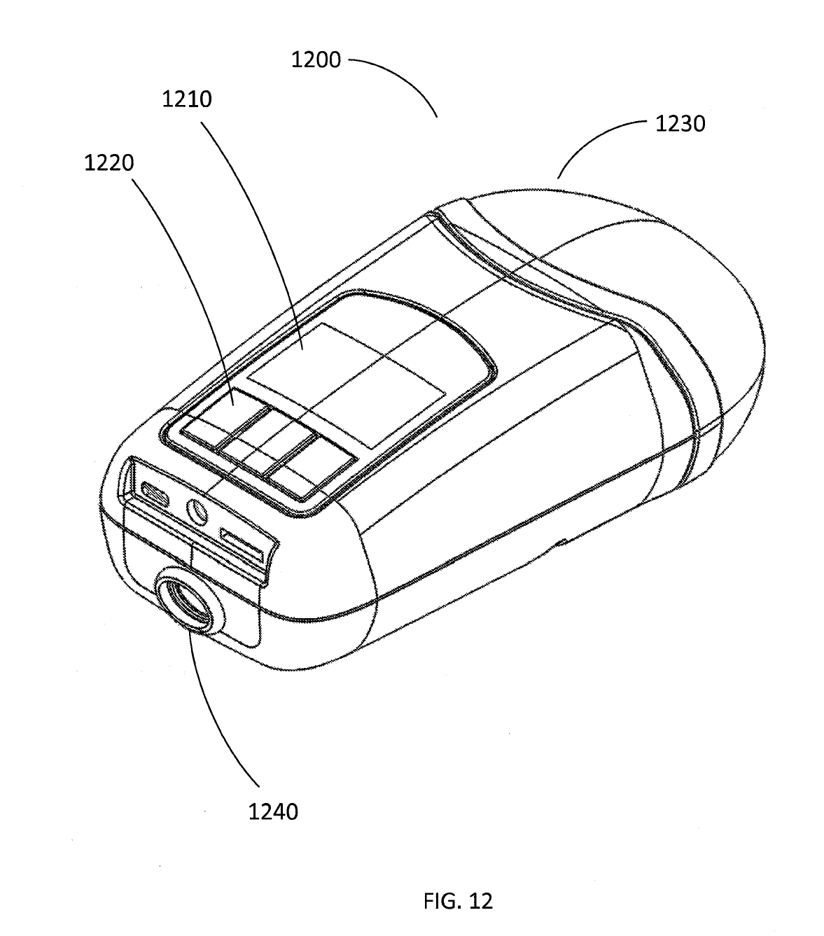

In a mode of operation, the user places the mask 26 on his I her face. In one mode, the user presses a button 64, as seen in FIG. 12, on the mask 26 and the system 20 goes into operation immediately. The mode of operation once the switch is pressed includes an open-loop mode or a closed-loop mode.

In another mode, the compressor 32 is not turned on until a later time. The later time can be based on a timer, detection of sleep, or detection of OSA. The time delay, detection of sleep, or detection of OSA to turn on the compressor 32 is described in U.S. patent application 61/559,912 filed on Nov. 15, 2011 which is incorporated herein by reference.

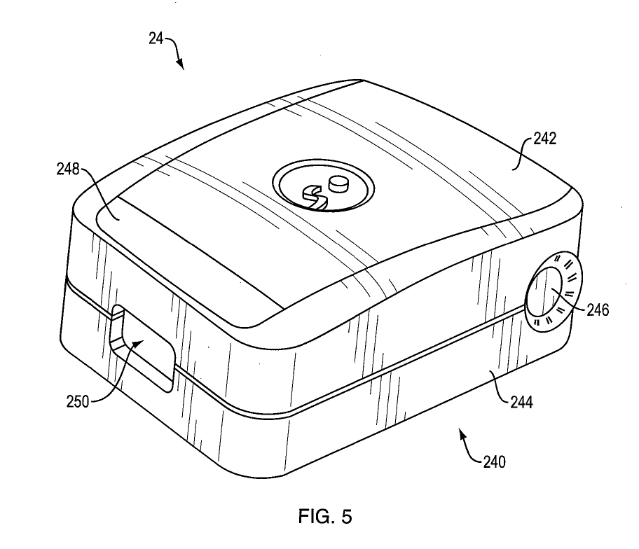

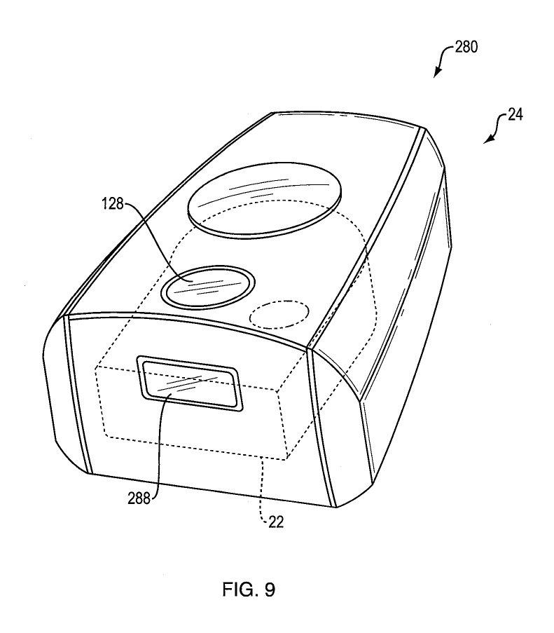

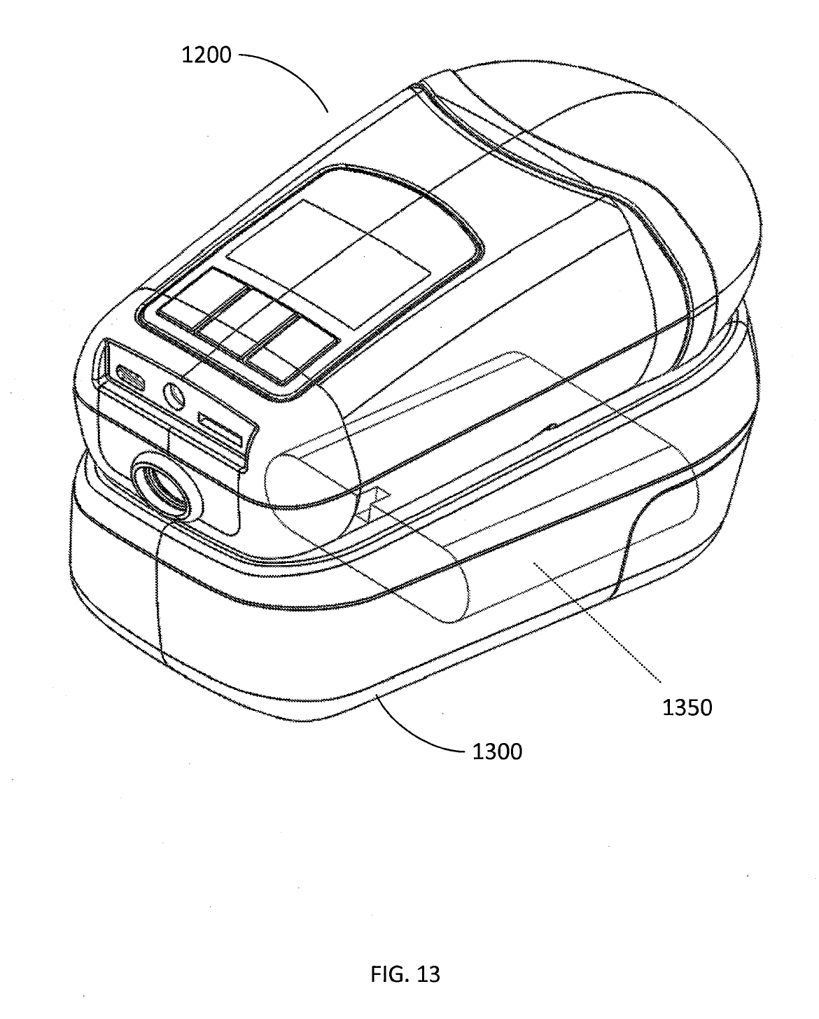

The first unit 22, the blower unit 22, can be accepted into various styles of the second unit 24. FIGS. 5-8B shows a clam shell docking station 240 that can be placed on a table. FIGS. 9-11 describe a second unit 24, a pouch 280 that a user can attach to their body. FIGS. 12-13 describe the blower unit 22 being placed into an integrated mask 300.

Referring back to FIGS. 3 and 4, the circuitry on the printed circuit board (PCB) 168 provides controlled output to the motor 160 that is used to rotate the impeller 118. In the embodiment shown in FIGS. 3 and 4, there are the pressure sensor 52, the pressure switch 62, which can be a pressure sensor, and the orientation sensor 198, which are used in the control of the compressor 32 in addition to the membrane switch 122. As indicated above, the membrane switch 122 shown in FIG. 2 has three input (membrane momentary) switches 128, 130, and 132 and four indicators in the forms of LEDs 134, 136, 138, and 140.

As indicated above, the first unit 22, the blower unit or flow generator 32, can be located at various locations. Referring to FIG. 5, a docking station 240, the second unit 24, that receives the first unit 22, the blower unit 22 is shown. The docking station 240 shown in perspective in FIG. 5 has a lower clam shell 242 and an upper clam shell 244 that enclose the blower unit 22. The docking station 240 has a hinge, a pivot point 246 that allows the upper clam shell 244 to pivot upward relative to the lower clam shell 242. The upper clam shell 244 in one embodiment has a single large button 248 for turning the compressor 32 on and off. The clam shells 242 and 244 of the docking station 240 form an opening 250 through which the hose 28 can connect to the blower unit 22 as shown in FIG. 7.

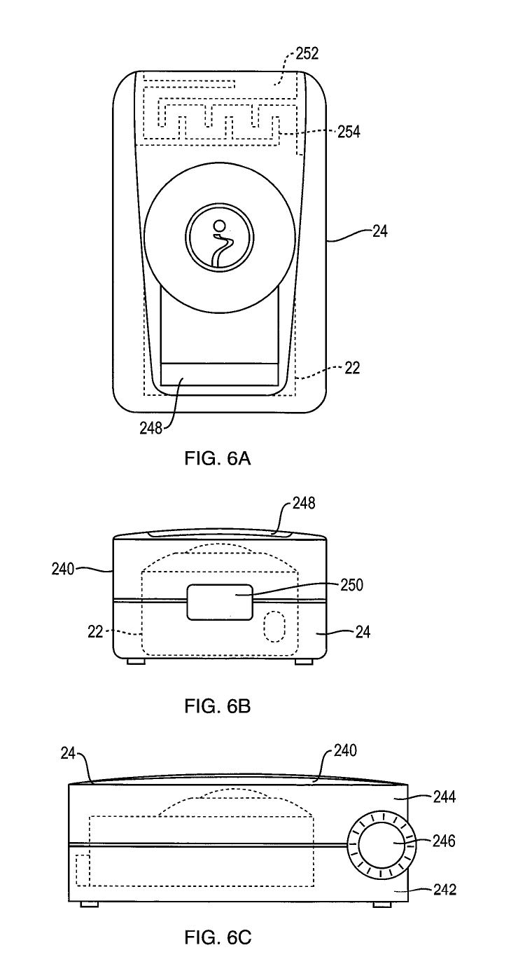

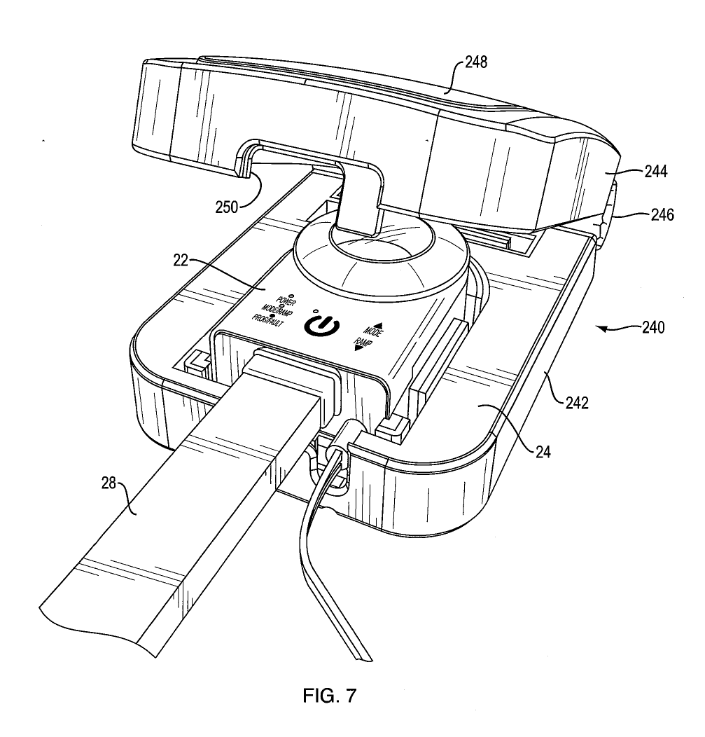

Referring to FIGS. 6A-6C, a top view, a front view, and a side view of the docking station 240 are shown. The top view, FIG. 6A, shows the first unit 24, the blower unit 24 in phantom line in the second unit 26, the docking station 240. In addition, a plurality of chambers 252 formed by baffling 254 in the docking station 240 are shown in hidden line. The baffling 254 is used to reduce the noise of the air being drawn into the compressor.

Referring to FIG. 7, a perspective view of the docking station 240 with the upper clam shell 244 hinged upward is shown. The hose 28 is shown passing through the opening 250 in the clam shells 242 and 244. A power cord connected to the first unit 22 extends through an opening in the second unit 24.

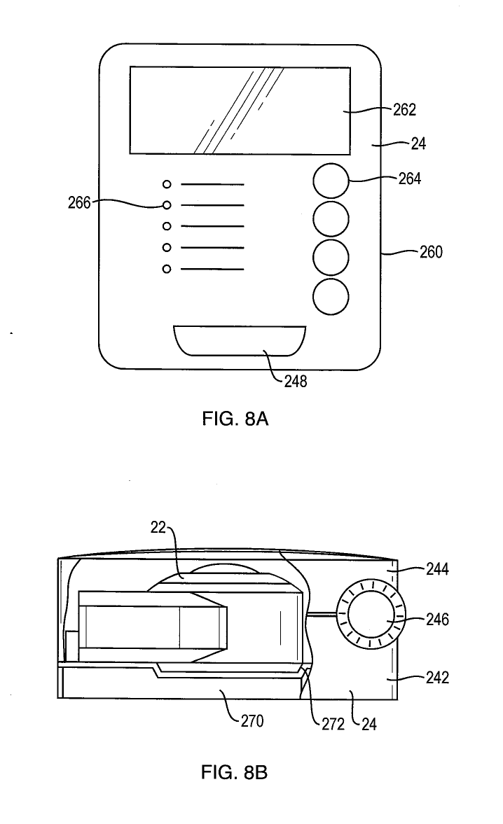

Referring to FIGS. 8A-B, a top view and a side view of an alternative second unit 24, the docking station 260 is shown. The top view shows a large on/off button 248 similar to the docking station 240 shown in FIGS. 5-7. In addition, the docking station 260 has a display 262, such as a color LCD, for displaying information such as mode, pressure, and RPM of the motor. In addition, the top of the docking station 260 has a plurality of additional switches 264 and indicator lights 266 for displaying additional information.

Referring to FIG. 8B, the side view of the alternative docking station 260 has a portion broken away. The docking station 260 in addition to being capable of being plugged into an electrical outlet, has a battery pack 270 that provides a back-up power source to the blower unit 22 that overlies the battery pack 270.

It is contemplated in certain models of the blower unit 22, that the blower unit 22 includes an internal power source. The blower unit 22 has a plurality of connectors 272 for receiving power, electricity, from the second unit.

In addition to placing the first unit 22 into a second unit 24 that is designed to be placed on a table, the first unit 22 can be placed in a second unit 24 that is designed to be carried on the body.

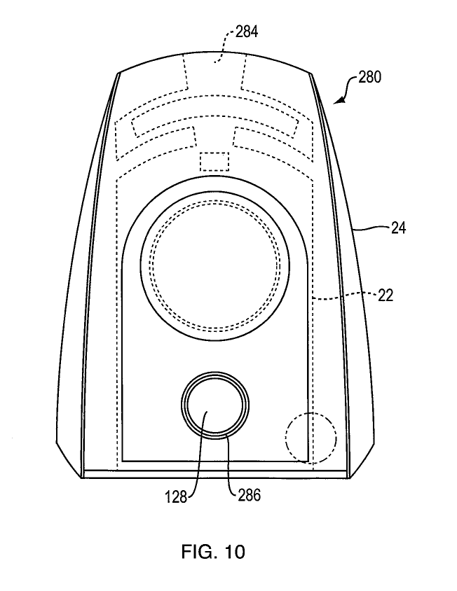

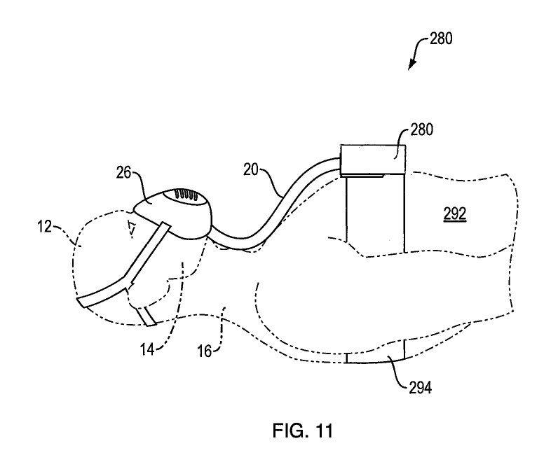

Referring to FIG. 9, a perspective view of a second unit 24 in this embodiment is a pouch 280 for accepting the blower or flow generator unit 22 is shown. The pouch 280 has several purposes including providing additional acoustic damping 282 as seen in FIG. 10, and providing padding therein allowing the blower unit 22 to be placed in locations such as strapped to the chest as seen in FIG. 11 or located in the bed and the user is able to make contact with the pouch 280 and not having to rest against the hard material of the blower unit 22.

Referring to FIG. 10, a top view of the pouch 280 for accepting the blower unit 22 is shown. Similar to the docking station 240, the pouch 280 has a series of baffles 284 to quiet the device. The pouch 280 has a large opening 286 to allow the user to gain access to the button 128 on the blower unit 22. The pouch 280 has an opening 288 through which the hose 28 passes, as best seen in FIG. 9.

As indicated above, the system 20 can be operated in several modes. In another mode or in combination with one or more modes above, the system has an orientation sensor 198 such as a tilt sensor or an accelerometer to determine the orientation of the system 20. The orientation sensor 198 can be located in the second unit 24 such as the mask 300 or in the first unit 22, the blower unit 22. As described above with respect to FIGS. 9-11, the location of the first unit 22 can be in various locations such as adjacent to the user's chest, lying next to the user such as on the bed mattress, or on a night stand or table adjacent to the bed. When the blower unit 22 is attached to the body of the user, such as affixed to the user's chest 292 by one or more straps 294 as shown in FIG. 11 or mounted to the mask 26 or straps 302 for the mask 26 as shown in FIG. 12, the orientation sensor 198 can be located in the blower unit 22 such as represented by the printed circuit board 168 in FIG. 8. In other situations such where the blower unit 22 is located on a night stand, the placement of the orientation sensor 198 in the mask 26 achieves a better result.

The orientation sensor 198 provides input to the controller 42 when the unit 22 is oriented in a vertical direction, such as when a user sits up or stands up. The system 20 can shut off the compressor 32 when the user is in this position. As indicated above, the user may choose not to select this mode for example if they are using the system while flying on a commercial airline.

In addition, the orientation sensor 198 in addition can determine if the user is lying on their back, stomach, or their side. In that the person's orientation effects the obstruction that causes sleep apnea, the amount of pressure needed varies from position to position.

In OSA, the upper airway collapses and blocks airflow during sleep. While the collapse can occur at several points, for example the soft palate in the upper oropharyngeal or pharynx level is drawn downward into the throat during sleep and blocks the airway, the orientation of the user and gravity effects can influence the percentage of blockage.

As indicated above, the first unit 22 can be placed in various forms of a second unit 24 which achieves multiple purposes including reduction of noise. As described shown in FIGS. 2 and 3 of PCT application PCT/US2010/053370 filed on Oct. 20, 2010, a detachable blower on mask system is contemplated and incorporated herein.

Another embodiment of a blower device is illustrated in FIG. 12. The portable blower 1200 is shown here configured with a screen 1210, interface buttons 1220, intake end 1230 with a screen covering the intake, and outlet 1240, which may be adaptable to a hose.

FIG. 13 illustrates portable blower 1200 docking with a cradle 1300 that has an internal power source 1350—such as a rechargeable battery. In some embodiments it is contemplated the portable blower device 1200 may be comprised of a minimum electronic components, a housing, an impellor and a motor drying the impellor. In such configurations, the controller, power source, memory and processing for auto-pap devices, may be contained in the second unit such as the cradle shown in FIG. 13, the mask disclosed in PCT/US2010/053370 filed on Oct. 20, 2010, or the docking station disclosed U.S. Ser. No. 13/450,614 filed Apr. 19, 2012.

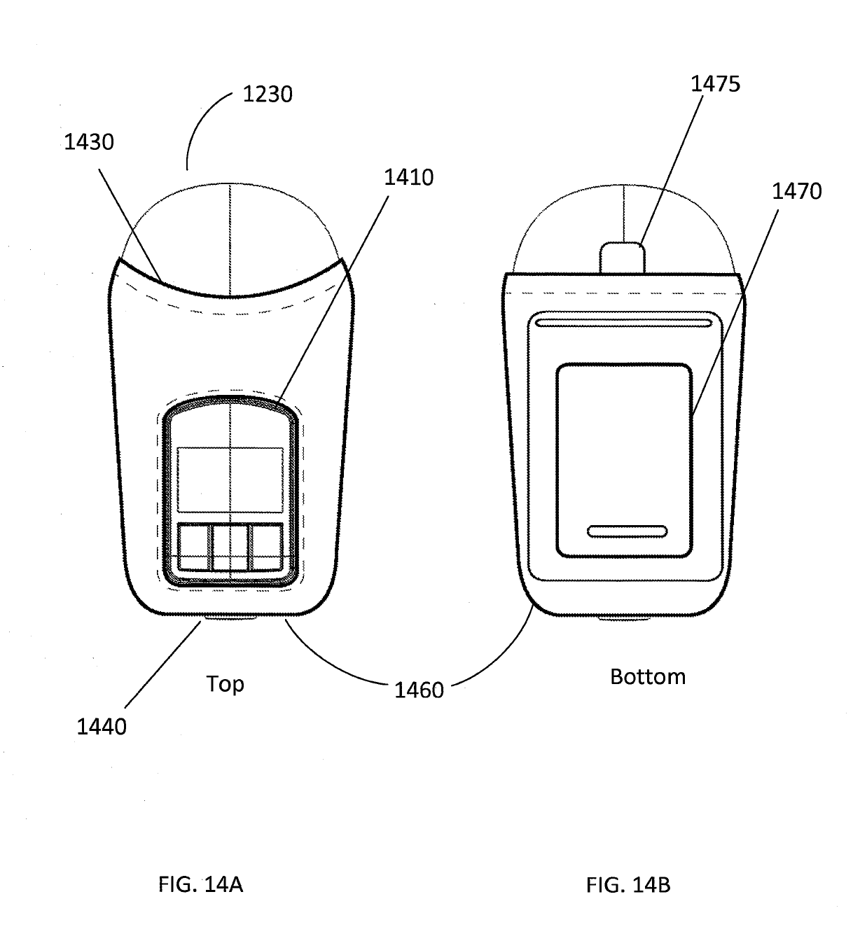

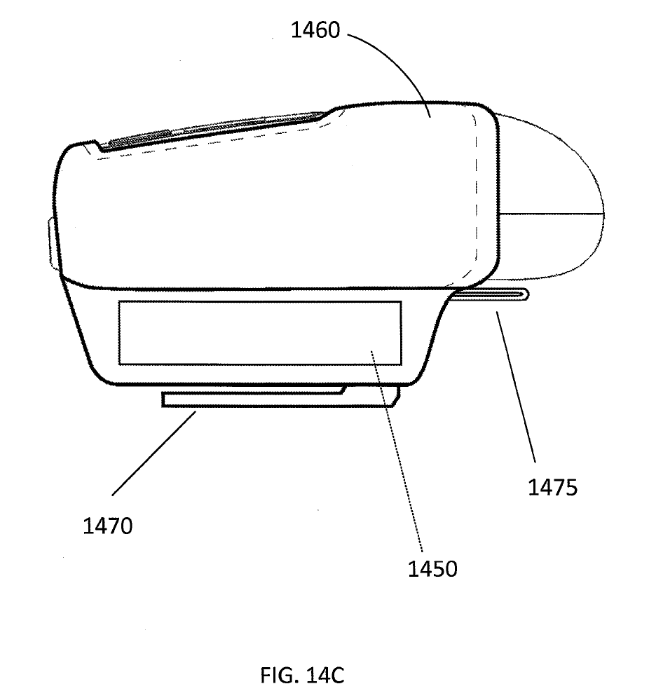

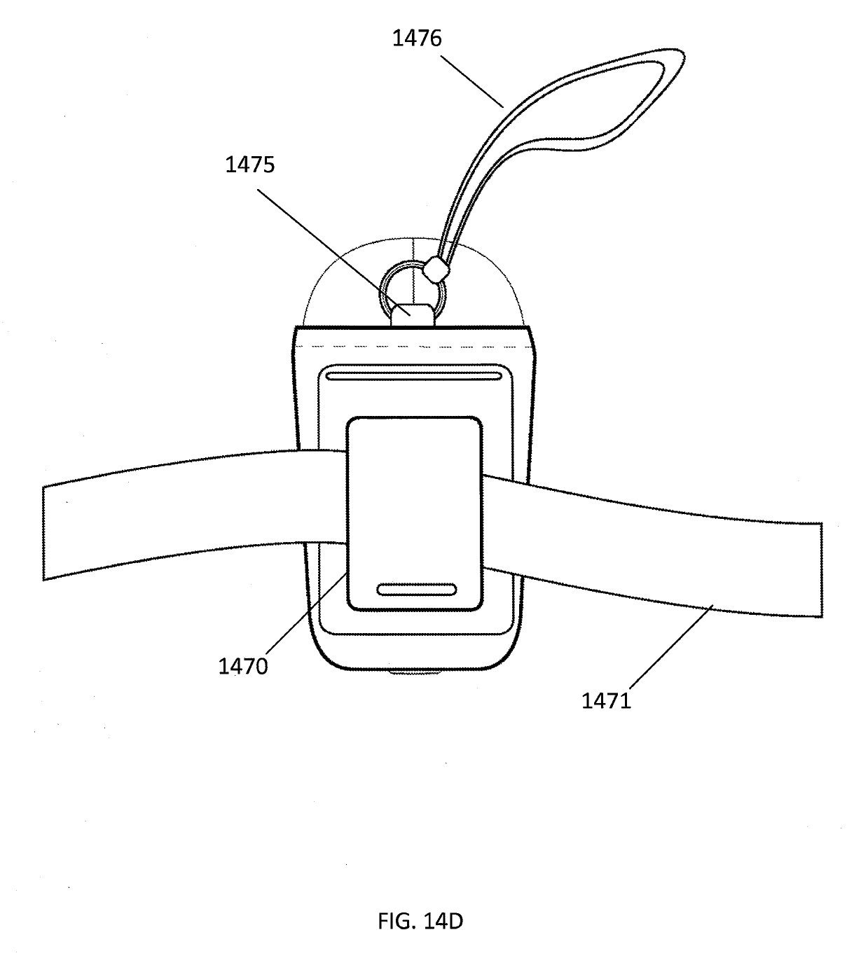

Additional pouch embodiments are disclosed in FIGS. 14A-D; however, here the pouch is designed to be supple and contain at least one attachment mechanism for convenient placement during use. FIGS. 14A-B illustrate a top and bottom view of a pouch that is configured to slide over a portable blower device 1200 with openings 1430 for air intake, 1440 for outlet, 1410 for viewing of the screen and access to interface buttons. Additionally a loop 1475 and clip 1470 are integrated into the pouch 1460 to allow the device to be hung by or over a bedside, connected to a belt loop, worn around a neck. The supple and soft housing allows the portable device to be used in bed and in close contact with the user, while not feeling like a hard mechanical device. As discussed above, and shown in FIG. 14C, pouch 1460 may have an integrated power source 1450 (and controller or additional acoustic dampening, etc.) built into the pouch 1460.

FIG. 14D illustrates pouch 1460 in use with a strap or belt 1471 and a lanyard 1476 or other device to hang the portable blower. This may be convenient to also help shorten the total length of the hose needed from the portable device to the mask worn by the user. It is also contemplated that other attachment mechanisms such as magnets, hook and loop material, snaps, buttons, handles and so forth may be integrated into the pouch embodiments described herein and those within the spirit and scope of this application. The soft shell or housing of 1460 may be made of a number of materials, including leather, synthetic materials, cotton, rubber or other textile materials. An inner hard structure for receiving a portable blower device may also exist to contain components such as a power source.

An integrated CPAP system contemplated herein is having a simple portable blower device having minimal components such as an impellor, motor, and housing that may be docked with any one of: a mask, docking station, hard or soft-shelled pouch. In this way a user/patient, may have the flexibility of using in a number of ways that are more ideal for comfort, portability, and convenience. The blower device or gas delivery system may also include sensors for detecting: pressure, fluid flow, motion, light and sound.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention.

1. A mounting unit for use with a gas delivery system comprising:
a housing having a cavity configured to receive a gas delivery system, wherein the housing has an inlet port, outlet port, and at least one attachment mechanism located on the outer portion of the housing.

2. The mounting unit of claim 1, wherein the housing is formed of a supple material.

3. The mounting unit of claim 1, further including a power source integrated at least partially within the housing.

4. The mounting unit of claim 1, further including an acoustic dampening pathway.

5. The mounting unit of claim 1, wherein the attachment mechanism is a clip.

6. The mounting unit of claim 1, further including a controller unit contained in the housing.

7. A mounting unit for use with a gas delivery system comprising:
a housing having a cavity configured to receive a gas delivery system, wherein the housing has an inlet port, outlet port, and power supply contained within at least a portion of the housing.

8. The mounting unit of claim 7, wherein the housing is formed of a supple material.

9. The mounting unit of claim 7, further including an attachment integrated into the outer portion of the housing.

10. The mounting unit of claim 7, further including an acoustic dampening pathway contained in the housing and configured to adapt to an intake of the gas delivery system.

11. The mounting unit of claim 9, wherein the attachment mechanism is a handle.

12. The mounting unit of claim 7, further including a controller unit contained in the housing.

13. The mounting of claim 7, wherein the housing encloses a majority of the gas delivery system.

14. The mounting of claim 7, wherein the power supply is a removable battery.

15. A detachable gas flow generator system comprising:
A first unit comprising a compressor that pressurizes gas, the compressor including an impellor and a motor, wherein the first unit has an inlet port and outlet for expelling compressed air;
and wherein the first unit may be received and mountable to at least one second unit comprised of the following: a mask, a pouch, or docking station, wherein the second unit has a power source configured to power the first unit.

16. The detachable gas flow generator system of claim 15, wherein the second unit has an acoustic dampening means.

17. The detachable gas flow generator system of claim 15, wherein the first unit has an orientation sensor.

18. The detachable gas flow generator system of claim 15, wherein the second unit contains an attachment means.

19. The detachable gas flow generator system of claim 15, further including at least two second units.

20. The detachable gas flow generator system of claim 15, further including three second units.
